(19) United States
(12) Patent Application Publication
(10) Pub. No.: US 2021/0386704 A1
(43) Pub. Date: Dec. 16, 2021

(54) MDMA RESPONSE PREDICTION

(71) Applicant: Universitätsspital Basel, Basel (CH)

(72) Inventors: Matthias Emanuel LIECHTI, Oberwill (CH); Patrick Raphael VIZELI, Kaiseraugst (CH)

(21) Appl. No.: 17/230,588

(22) Filed: Apr. 14, 2021

Related U.S. Application Data
(60) Provisional application No. 63/039,131, filed on Jun. 15, 2020.

(51) Int. Cl. A61K 31/36; G16H 10/20; G16H 20/10; G16H 70/40; G16H 10/60

(52) U.S. Cl. CPC A61K 31/36; G16H 10/20; G16H 10/60; G16H 70/40; G16H 20/10

(57) ABSTRACT

A method of dosing an empathogen/entactogen in treating patients, by assessing patient characteristics before empathogen/entactogen use, administering empathogen/entactogen to the patient based on the patient characteristics, and producing maximum positive subjective acute effects in the patient. A method of determining a dose of an empathogen/entactogen based on body weight, sex, and CYP2D6 activity by assessing patient characteristics of body weight, sex, and CYP2D6 activity before empathogen/entactogen use, administering the empathogen/entactogen to the patient based on the patient characteristics, and producing maximum positive subjective acute effects in the patient. A method of refining dosing of an empathogen/entactogen. A method of predicting future dosing with an empathogen/entactogen. A method of evaluating feasibility of patients to receive an empathogen/entactogen as treatment. A method of optimizing empathogen/entactogen treatment in a patient.

### BACKGROUND OF THE INVENTION

### 1. Technical Field

The present invention relates to methods for predicting the response to (±)3,4-methylenedioxymethamphetamine (MDMA) to optimize the induction of specific acute effects in treating medical conditions.

### 2. Background Art

MDMA is a psychoactive drug that alters mood and perception and is investigated as an adjunct in psychotherapy for posttraumatic stress disorder and may later also be studied for a range of other medical conditions (Mithoefer et al., 2019; Mithoefer et al., 2010; Oehen et al., 2013).

It is unclear what the ideal dose of MDMA is in this therapeutic modality. Specifically, the dose may vary depending on many factors including but not limited to sex, weight, age, metabolic differences, state of mood and mind before MDMA administration, and personality trait factors. Therefore, finding the correct dose of MDMA in an individual is a problem.

While MDMA acutely induces mostly positive subjective effects including heightened mood, openness, trust, and enhanced empathy, there can also be negative drug effects (Hysek et al., 2014a; Schmid et al., 2014).

Some previous studies have defined single variables that influence the response to MDMA. The variables influencing a drug experience are classically grouped into set and setting (Leary et al., 1963). The set consists of factors related to the person such as personality, age, and previous drug experiences as well as the person's expectations and intentions regarding the drug effect. The setting relates to environmental factors such as the place of substance intake, the persons present at time of intake, and cultural surroundings (Hartogsohn, 2016).

The drug effects of MDMA have been shown to be dose dependent (Bedi & de Wit, 2011; Vizeli & Liechti, 2017). However, it is unclear how additional factors such as set and setting can be used in addition to dose to help predict the individual response to MDMA.

For other substances such as psilocybin, acute effects were found to be mostly dose dependent (Haijen et al., 2018; Studerus et al., 2012). Additional variables such as personality traits and mood before drug intake were also found to influence the acute effects of psilocybin (Haijen et al., 2018; Studerus et al., 2012).

“Bad trips” (also referred to as “challenging experiences” when moderate) are of special interest when using psychoactive substances including MDMA. Personality traits may predict such bad trips. For example, “neuroticism” which reflects a person's emotional instability and poor coping with stressful events (Ormel et al., 2012) was shown to be associated with a “challenging experience” after taking psilocybin (Barrett et al., 2017).

A number of previous studies investigated potential pharmacological and non-pharmacological factors contributing to the response to MDMA, including sex (Bedi & de Wit, 2011; Liechti et al., 2001; Pardo-Lozano et al., 2012; Simmler et al., 2011), pharmacokinetics (Pardo-Lozano et al., 2012; Vizeli et al., 2017), personality traits (White, 2017), and genetics (Bershad et al., 2016; Schmid et al., 2016; Vizeli & Liechti, 2018; Vizeli et al., 2018; Vizeli et al., 2019; Vizeli et al., 2017).

However, all of these studies each assessed only a small number of potential predictors, did not adjust for potentially confounding variables, and did not assess the importance of the different variables. Additionally, the study sample sizes of the previous studies were rather small and could not enable such analyses. Therefore, there remains a need for methods of predicting responses to MDMA that take multiple variables into account.

### SUMMARY OF THE INVENTION

The present invention provides for a method of dosing an empathogen/entactogen (such as MDMA) in treating patients, by assessing patient characteristics before empathogen/entactogen use, administering the empathogen/entactogen to the patient based on the patient characteristics, and producing maximum positive subjective acute effects in the patient.

The present invention provides for a method of determining a dose of an empathogen/entactogen based on body weight, sex, and CYP2D6 activity, by assessing patient characteristics of body weight, sex, and CYP2D6 activity before empathogen/entactogen use, administering the empathogen/entactogen to the patient based on the patient characteristics, and producing maximum beneficial subjective acute effects in the patient.

The present invention also provides for a method of refining dosing of an empathogen/entactogen by using questionnaires including NEO-FFI, STAI-T, and AMRS scales with a patient, evaluating questionnaire responses, and refining dosing of the empathogen/entactogen in the patient based on the questionnaires.

The present invention provides for a method of predicting future dosing with an empathogen/entactogen, by measuring plasma concentrations of an empathogen/entactogen in a patient after administration of a dose of the empathogen/entactogen, adjusting the dose of the empathogen/entactogen in the patient, thereby optimizing the positive response to the empathogen/entactogen, and optimizing efficacy and safety of the empathogen/entactogen treatment.

The present invention provides for a method of evaluating feasibility of patients to receive an empathogen/entactogen as treatment, by assessing patient characteristics, and evaluating feasibility of the patient to receive the empathogen/entactogen as a treatment.

The present invention further provides for a method of optimizing empathogen/entactogen treatment in a patient, by assessing openness in the patient prior to empathogen/entactogen use, predicting a positive response leading to more openness, and optimizing empathogen/entactogen treatment including repeated administration to lead to greater openness and a greater therapeutic response over time.

### DESCRIPTION OF THE DRAWINGS

Other advantages of the present invention are readily appreciated as the same becomes better understood by reference to the following detailed description when considered in connection with the accompanying drawings wherein:

FIG. 1 is a graph showing unadjusted linear mixed effects models with MDMA plasma concentration as predictor and response variable, smaller asterisks show non-corrected p-values, bigger asterisks are p-values corrected for multiple testing across all 20*25=500 significance tests using the Benjamini-Hochberg procedure (Benjamini & Hochberg, 1995). *p<0.05, **p<0.01, ***p<0.001;

FIG. 2 is a graph showing a mixed effects model with variables corrected for dose/bodyweight, smaller asterisks show non-corrected p-values, bigger asterisks are p-values corrected for multiple testing across all 17*25=425 significance tests using the Benjamini-Hochberg procedure (Benjamini & Hochberg, 1995). *p<0.05, **p<0.01, ***p<0.001; and

FIG. 3 is a graph of predictor values and effects of MDMA.

### DETAILED DESCRIPTION OF THE INVENTION

The present invention generally provides for methods of predicting response to empathogen/entactogen (such as MDMA) treatment by analyzing various predictor variables in patients. More specifically, a method is provided of dosing an empathogen/entactogen in treating patients by assessing patient characteristics before empathogen/entactogen use, administering the empathogen/entactogen to the patient in a therapeutic situation or in a legal controlled situation in healthy subjects including but not limited to a clinical study, a use to train therapists, or any other legal controlled setting in healthy subjects, and producing maximum positive subjective acute effects in the patient. This method can be used to better target the dose range in an individual to more likely produce a positive acute response to empathogens/entactogens.

“Positive subjective acute effects” as used herein refers to any desired effects of the MDMA, such as, but not limited to, self-ratings on a visual analog scale including “good drug effects”, “drug liking”, “trust”, “feelings of closeness”, “feeling open”, 5D-ASC scale or Mystical Effects scale ratings or similar of oceanic boundlessness, experience of unity, spiritual experience, blissful state, insightfulness, connectedness, mystical experiences, mystical-type effects, positive mood, transcendence of time/space, ineffability, well-being, and peak experience.

The present invention provides for a solution of the dosing problem experienced in the prior art by the characterization of predictor variables that can be used for correct dosing and optimizing dosing and allows for better and safer selection of patients and dosing in patients to be treated with MDMA. The present invention uses a large data set (as opposed to the prior art which used only single variables and a small data set) to define the influence of multiple variables and their interdependence to derive dosing recommendations and predictors for the physiological and psychological response to MDMA. The present invention is based on data of ten controlled experimental studies with a total sample size of 194 healthy subjects tested in the same laboratory over a 10-year period.

The predictor variables/patient characteristics can include, but are not limited to, age, sex, drug dose, body weight, previous drug experience, genetics, personality, and mood before intake. From the studies, several predictor variables were found to be the most important in predicting effects.

While MDMA is referred to herein, it should be understood that any empathogen/entactogen or MDMA-like compound such as but not limited to 3,4-methylendioxyamphetamine (MDA), 3,4,-methylenedioxyethylamphetamine (MDEA), 5,6-methylenedioxy-2-aminoindane (MDAI), mephedrone, methylone, 3-MMC, homologues thereof, analogues thereof, or novel compounds or prodrugs resulting in a similar MDMA-type acute subjective effect profile can be used in the methods herein. Any other compound that provides a similar MDMA-type acute subjective effect profile can also be used. In the methods herein, MDMA is preferably administered in a dose of 20-200 mg.

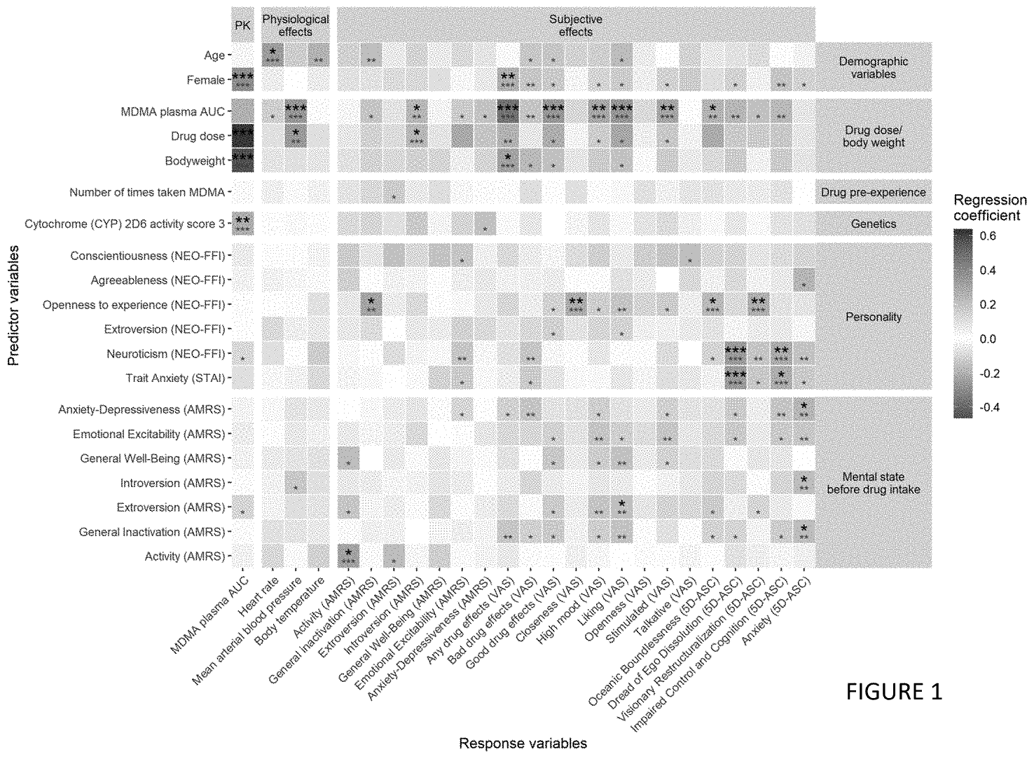

MDMA plasma concentration after its administration to the individual and its proxy variable dose per kg body weight are the most important predictors of the acute response to MDMA and the intensity of the response to MDMA. The dose of MDMA per body weight is a surrogate measure for the plasma concentration of MDMA. This surrogate measure is known before MDMA administration and can be used for correct dosing. A high dose of MDMA per body weight (within the 75-125 mg absolute dose range) leads to a more intense response to MDMA with more positive and more cardiostimulant effects (as shown in FIG. 1).

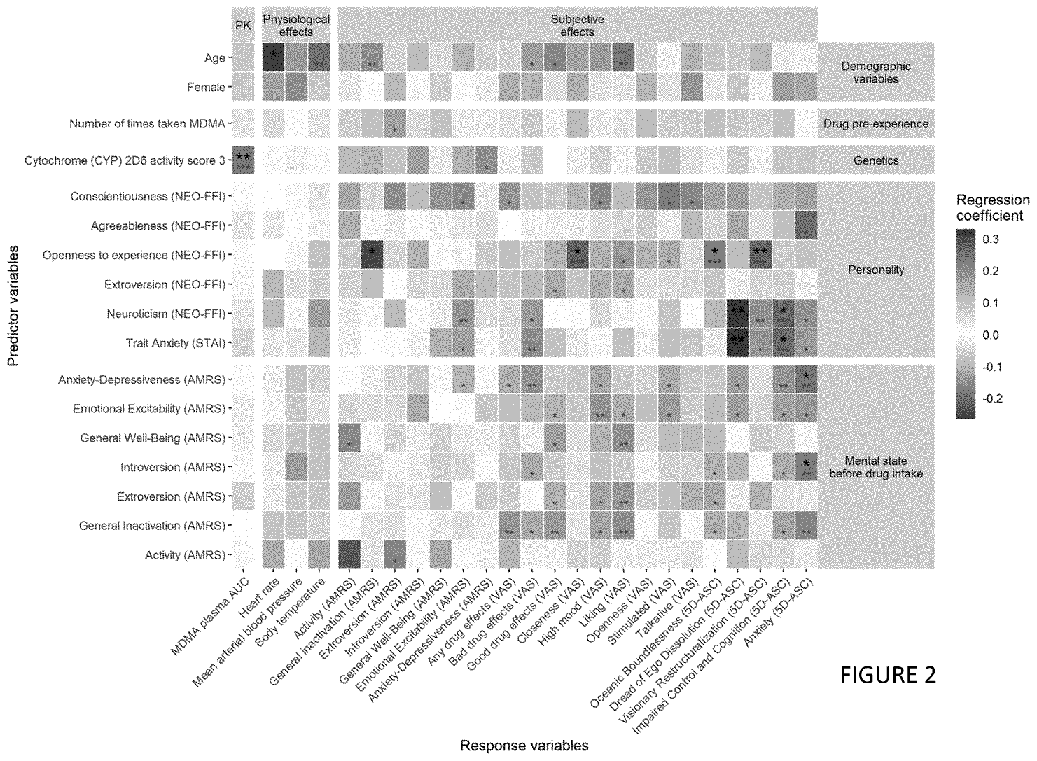

Dosing does not have to account for sex of the individual if body weight is accounted for (FIG. 2). However, some sex differences in the MDMA experience were previously reported in controlled studies even after adjusting for differences in dosing (Liechti et al., 2001; Simmler et al., 2011; Vizeli & Liechti, 2017). If dosing is performed independent of body weight, women need to be dosed approximately 20-30% lower than men (125 mg in men, 100 mg in women) to account for body weight differences between sexes.

Cytochrome P450 (CYP) 2D6 activity (genetically or phenotypically determined; Schmid et al., 2016). influences plasma concentration of MDMA (FIGS. 1 and 2). Subjects with poor CYP2D6 activity exhibit higher MDMA concentrations and a stronger MDMA response compared with CYP2D6 extensive metabolizers. In line with this finding, CYP2D6 poor metabolizers have previously been shown to have higher MDMA blood plasma concentrations than extensive/normal metabolizers (de la Torre et al., 2005; Schmid et al., 2016). The present invention in addition allows defining doses in patients with different CYP2D6 functions and suggesting dose reductions of MDMA for subject with CYP2D6 poor metabolizer status. For example, a dose of 100 mg instead of 125 mg would be used in a male poor metabolizer and a dose of 80 mg instead of 100 mg would be used in a female patient when MDMA is first used in such a patient with known 2D6 activity and based on the present invention and without taking into account additional dosing parameters.

Therefore, the present invention provides for a method of determining a dose of MDMA based on body weight, sex, and CYP2D6 activity, by assessing patient characteristics of body weight, sex, and CYP2D6 activity before MDMA use, administering MDMA to the patient, and producing maximum positive subjective acute effects in the patient.

Psychological factors such as “openness to new experiences”, “neuroticism”, or “trait anxiety” are significant predictors for the acute subjective drug effects of MDMA after adjusting for dose/kg.

The personality trait “openness” enables increased “feelings of closeness” in response to MDMA strengthening the therapeutic alliance. “Openness to experience” (NEO-FFI) predicts higher “closeness” (VAS) after MDMA (FIG. 2). This prediction is in line with greater good drug effects in persons with greater sensation seeking (Harvanko et al., 2016) which is linked to “openness to experience” (Roberti, 2004). Closeness and openness after MDMA was found to be linked to reductions in PTSD symptoms (Wagner et al., 2017).

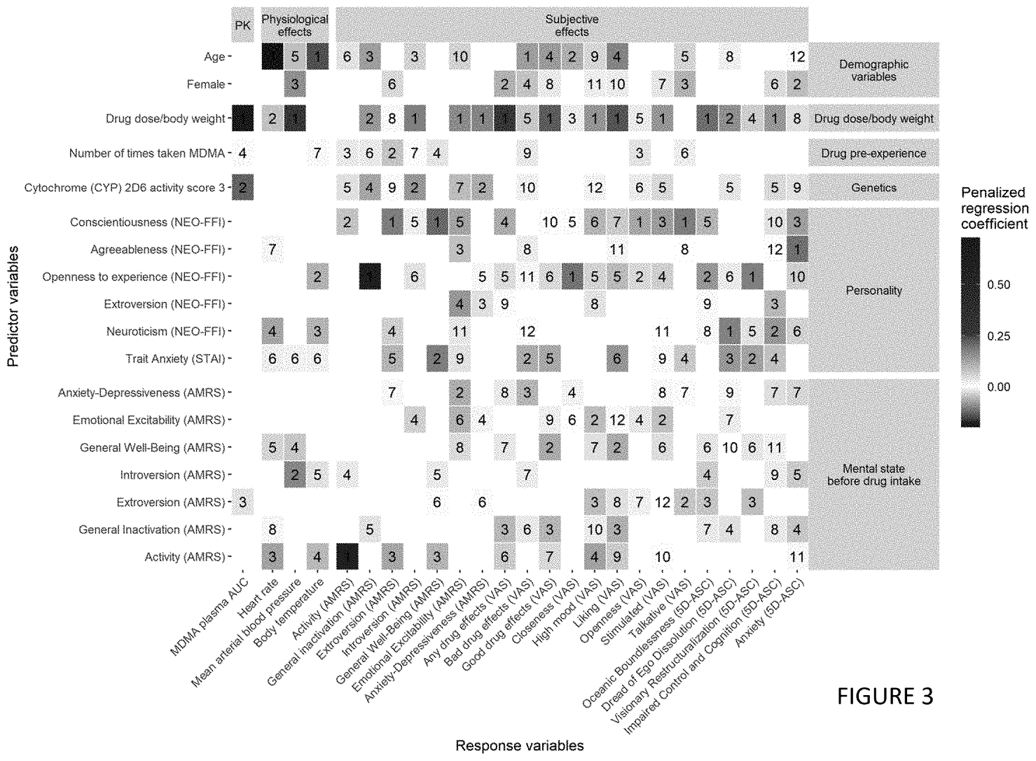

“Openness to experience” (NEO-FFI) predicts higher 5D-ASC ratings of “visionary restructuralization” after MDMA (FIG. 2). Consistently, “absorption”/openness predicted visual effects of psychedelics (Haijen et al., 2018). Also, consistently, “openness to experience” was the strongest predictor of feeling “closeness” (VAS) and having high 5D-ASC ratings of “visionary restructuralization” after MDMA (FIG. 3).

Subjects scoring high on “trait anxiety” and “neuroticism” experience more negative MDMA effects. These personality traits likely lead to more fear of loss of control.

Higher “neuroticism” (NEO-FFI) predicts greater anxiety (“dread of ego dissolution” in the 5D-ASC) (FIG. 2). Consistently, neuroticism (NEO-FFI) was the best predictor of elevated dread of ego dissolution ratings in the 5D-ASC after MDMA (FIG. 3).

Consistently, more pronounced “neuroticism” was linked to more “challenging experiences” after taking psychedelic substances (Barrett et al., 2017; Haijen et al., 2018; Studerus et al., 2012).

Higher “trait anxiety” (STAI-T) predicts greater anxiety (“dread of ego dissolution” in the 5D-ASC) (FIG. 2).

Previous MDMA experience showed no effect on the response to MDMA, consistent with another study (Bedi & de Wit, 2011). However, it is important to note that the present data set includes mostly subjects who were MDMA naïve or had a maximum of only five previous MDMA experiences. Therefore, the influence of heavier past MDMA use could not be assessed. Notably, patients in clinical trials using MDMA will, similar to the present study population, likely have no to little experience in using MDMA.

The method can be used to design a score to predict the response to MDMA.

The method can be used to predict positive responses to MDMA in future sessions or to adjust dosing based on measuring concentrations of MDMA in one session.

The method can be used to further optimize or predict the response to MDMA once the perfect dose of MDMA has been determined based on body weight and CYP2D6 status (and sex). The method can also be used to find the optimal dose once a patient is selected to be suitable for a treatment with MDMA.

Important predictors for the response, once the MDMA dose has been determined or to determine the feasibility of a potential patient to receive MDMA, are:

Openness to experience (NEO-FFI) which predicts a more positive acute response including greater closeness, Oceanic Boundlessness (5D-ASC), and Visionary Restructuralization (5D-ASC) (FIG. 2);

Neuroticism (NEO-FFI) which predicts negative experiences including Dread of Ego-Dissolution (5D-ASC) and Impaired Control and Cognition (5D-ASC) (FIG. 2);

Trait anxiety (STAI) which predicts negative experiences including Dread of Ego-Dissolution (5D-ASC) and Impaired Control and Cognition (5D-ASC) (FIG. 2).

Thus, the NEO-FFI and STAI can be used prior to a session to form a score to predict the likelihood of a positive and/or negative acute effect of MDMA during the session.

Further, mood before intake including the following can be used as a predictor:

anxiety/depressiveness (AMRS) predict anxiety (5D-ASC) (FIG. 2);

introversion (AMRS) predict anxiety (5D-ASC) (FIG. 2).

Thus, the AMRS can be used before MDMA administration to predict the likelihood of a negative acute effect of MDMA including anxiety during the session.

Therefore, the present invention provides for a method of refining dosing of MDMA, by using questionnaires including NEO-FFI, STAI-T, and AMRS scales with a patient, evaluating the questionnaire responses, and refining dosing of MDMA for the patient based on the questionnaires. For example, reduced doses of MDMA are recommended in patients with low NEO-FFI openness ratings, high neuroticism NEO-FFI ratings, high STAI-trait anxiety ratings. High anxiety-depressiveness (AMRS) and introversion (AMRS) ratings before drug intake predict higher anxiety after MDMA and a dose reduction would also be advised based on the present invention and subject to further refinement and implementation of the present application.

Taken together, the present invention makes use of dosing parameters such as dose of MDMA per body weight, CYP2D6 genotype, data derived from the NEO-FFI and STAI as well as AMRS to optimize the dosing and predict more positive over negative experiences with MDMA to thereby increase the safety and effectiveness of its administration in patients.

A study in PTSD patients found a relationship between reduction of PTSD symptoms and increased “openness” after MDMA treatment (Wagner et al., 2017). The traits “openness” and “neuroticism” were increased and reduced after MDMA treatment, respectively. A similar persisting effect on personality traits was also observed after use of psilocybin or LSD (MacLean et al., 2011; Schmid & Liechti, 2018).

Further, because MDMA increases openness and because openness predicts a more positive response to MDMA including greater therapeutic effects, this indicates that patients might progressively benefit from multiple MDMA-assisted psychotherapy sessions, as they likely become more open to the experience over time.

Therefore, the present invention provides for a method of optimizing MDMA treatment in a patient, by assessing openness in the patient prior to MDMA use, predicting a positive response leading to more openness, and optimizing MDMA treatment including repeated administration to lead to greater openness and a greater therapeutic response over time.

The present invention also provides for a method of predicting future dosing with MDMA, by measuring plasma concentrations of MDMA in a patient after administration of a dose of MDMA, adjusting the dose of MDMA in the patient, thereby optimizing the positive response to MDMA, and optimizing efficacy and safety of MDMA treatment.

The present invention provides for a method of evaluating feasibility of patients to receive MDMA as treatment by assessing patient characteristics and evaluating feasibility of the patient to receive MDMA as a treatment.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

Materials and Methods

Study Design

A pooled analysis was performed of the raw data from ten double-blind, placebo-controlled, crossover studies in healthy human subjects, of all of which have previously been described (Dolder et al., 2018; Holze et al., 2020; Hysek et al., 2012a; Hysek & Liechti, 2012; Hysek et al., 2012b; Hysek et al., 2011; Hysek et al., 2012c; Hysek et al., 2014b; Schmid et al., 2015a; Schmid et al., 2015b).

The studies were conducted at the University Hospital Basel from 2009 to 2018 and include a total of 194 healthy subjects. Seven studies each included 16 subjects (total of 112 subjects) who received 125 mg MDMA twice, once alone and once after pretreatment with a medication (Hysek et al., 2012a; Hysek & Liechti, 2012; Hysek et al., 2012b; Hysek et al., 2011; Hysek et al., 2012c; Hysek et al., 2014b; Schmid et al., 2015b). In three additional studies, subjects received MDMA alone, placebo, and one or two other substances (Dolder et al., 2018; Holze et al., 2020; Schmid et al., 2015a). Of these, one used an MDMA dose of 75 mg (n=30) (Schmid et al., 2015a) and the others used 125 mg (n=24 and n=28) (Dolder et al., 2018; Holze et al., 2020).

In the present analysis, only data from the MDMA-alone and placebo sessions were used. In all of the pooled studies, the washout periods between the single-dose administrations of MDMA were at least 7 days to exclude carry-over effects. The studies were all registered at ClinicalTrials.gov (NCT00886886, NCT00990067, NCT01136278, NCT01270672, NCT01386177, NCT01465685, NCT01771874, NCT01951508, NCT01616407, NCT03019822).

Detailed pharmacokinetic and safety data from these studies have been published elsewhere (Schmid et al., 2016; Vizeli & Liechti, 2017; Vizeli et al., 2017).

Test sessions were conducted in a quiet hospital research ward with no more than two research subjects present per session. The participants were comfortably lying in hospital beds and were mostly listening to music and not engaging in physical activities. MDMA was administered without food in the fasting state in the morning at 8:00-9:00 AM. A small, standardized lunch was served at 12:00-1:00 PM.

Participants

A total of 194 (97 female) healthy subjects, aged 18-45 years (mean±SD=25.1±4 years) participated in the study. One genotyping sample was missing, and three participants did not give consent for genotyping. The mean±SD body weight was 69±10 kg (range: 46-97 kg). Exclusion criteria included a history of psychiatric disorders, physical illness, a lifetime history of illicit drug use more than ten times (with the exception of past cannabis use), illicit drug use within the past 2 months, and illicit drug use during the study. Drug screens were conducted before the test sessions as reported in detail elsewhere (Hysek et al., 2012a; Hysek & Liechti, 2012; Hysek et al., 2012b; Hysek et al., 2012c). Seventy-five subjects had prior illicit substance experiences (1-8 times), of which 41 subjects had previously used MDMA (1-5 times), 18 subjects had previously used amphetamine or methamphetamine (1-2 times), 15 subjects had previously used cocaine (1-4 times), 10 subjects had previously used lysergic acid diethylamide (1-2 times), and 15 subjects had previously used psilocybin (1-4 times).

Study Drug

(±)MDMA hydrochloride (Lipomed AG, Arlesheim, Switzerland) was administered orally at a single dose of 75 or 125 mg prepared as gelatin capsules. Male and female subjects received the same doses of MDMA irrespective of their body weight as it is done in therapeutic studies (Mithoefer et al., 2010; Oehen et al., 2013). The dose per body weight (mean±SD) was 1.7±0.4 mg/kg (range: 0.8-2.7 mg/kg).

Predictor Variables

Effects of MDMA were expected to be dose- and body weight-dependent (Schmid et al., 2014; Vizeli & Liechti, 2017). Therefore, dose divided by body weight was included as covariate in the analysis. This also accounted for the higher mg/kg dose of MDMA in females compared with males due to the lower body weight in women compared with men.

From the socio-demographic predictor variable domain, sex and age as predictors were included. Sex was included because sex differences in the MDMA experience were reported in several controlled studies even after adjusting for differences in dosing (Liechti et al., 2001; Simmler et al., 2011; Vizeli & Liechti, 2017).

Age was included since younger age was associated with more unpleasant acute effects of psilocybin (Studerus et al., 2012), while no data is available on MDMA.

Individual metabolic differences in the enzymes metabolizing MDMA influence the exposure to MDMA and thereby its acute effects. Specifically, the activity of cytochrome P450 enzymes has been shown to alter MDMA concentrations and concomitant subjective and cardiovascular responses (de la Torre et al., 2012; Schmid et al., 2016; Vizeli et al., 2017). Thus, the CYP2D6 genetic activity score (Hicks et al., 2013; Schmid et al., 2016) was included as an additional predictor variable.

Measures of other CYP enzyme activity were not included as these have been shown to have no or only very small effects on the response to MDMA (Vizeli et al., 2017). Likewise, other potential pharmacogenetic predictors were not included, because they also showed no or only minimal effects on the acute response to MDMA (Bershad et al., 2016; Vizeli & Liechti, 2018; Vizeli et al., 2018; Vizeli et al., 2019).

Although all subjects had no or only very limited previous experiences with psychoactive substances (0-5 times), the number of MDMA consumptions prior to participation was included in the analysis, since MDMA effects have been reported to change with long-term use and more experienced users experienced smaller drug effects than inexperienced persons (Kirkpatrick et al., 2014). The present study could not address potential effects of high previous substance use (>5 times) or the moderating effects of physical activity on the cardiovascular and thermogenic response to MDMA because persons with extensive previous drug experiences were not included and all subjects were physically inactive during the drug response (Liechti, 2014).

Mood states prior to the administration of a psychoactive substance may influence its response as previously shown for psilocybin in a similar study (Studerus et al., 2012). Therefore, ratings on the Adjective Mood Rating Scale (AMRS) (Janke & Debus, 1978) were included to assess mood states prior to the MDMA administration. Sixty adjectives were rated on 4-point Likert scales and items were grouped into six main scales: ‘Performance-Related Activity’, ‘General Inactivation’, ‘Extroversion-Introversion’, ‘General Well-Being’, ‘Emotional Excitability’, and ‘Anxiety-Depressiveness’. ‘Extraversion’ and ‘Introversion’ were analyzed separately.

Personality traits were assessed using the NEO-FFI (Borkenau & Ostendorf, 2008) which contains 60 self-referent statements rated on a 4-point Likert scale. The NEO-FFI covers the personality traits ‘Neuroticism’, ‘Extraversion’, ‘Openness to experience’, ‘Agreeableness’ and ‘Conscientiousness’. Subjects completed the questionnaire as part of the screening procedure at the start of the study. Finally, the trait scale of the State-Trait Anxiety Inventory (STAI-T) was included (Spielberger et al., 1970). This self-assessment questionnaire contains 20 statements describing anxiety as a stable personality trait.

Response Variables

Blood samples for the pharmacokinetic response were collected in lithium heparin tubes 0, 0.33, 0.67, 1, 1.5, 2, 3, 4, and 6 hours after administration of MDMA or placebo and immediately centrifuged.

Plasma was stored at −20° C. until analysis. Plasma concentrations of MDMA were determined as previously described (Hysek et al., 2012c). The area under the concentration-time curve (AUC) from 0 to 6 hours after dosing was calculated following the trapezoidal rule as a measure of total exposure to MDMA.

The subjective response to MDMA was assessed using psychometric scales. Visual Analog Scales (VASs) (Hysek et al., 2011; Hysek et al., 2012c) were used before and 0.33, 0.67, 1, 1.5, 2, 2.5, 3, 4, 5 and 6 hours after MDMA or placebo administration. VASs for “any drug effect”, “good drug effect”, “bad drug effect”, “high mood”, “drug liking”, and “stimulated” were presented as 100-mm horizontal lines (0-100%), marked from “not at all” on the left to “extremely” on the right. The VASs “closeness”, “concentration”, “openness”, and “talkative” were bidirectional (±50%). Additionally, the AMRS was administered 1.25, 2, and 5 hours after administration of MDMA or placebo. The response on each VAS and AMRS subscale was included into the analysis as area under the effect-time-curve (AUEC) value, reflecting the overall response throughout the study day.

Blood pressure, heart rate, and body temperature were assessed repeatedly before and 0, 0.33, 0.67, 1, 1.5, 2, 2.5, 3, 4, 5, and 6 hours after MDMA or placebo administration. Systolic and diastolic blood pressure and heart rate were measured using an automatic oscillometric device (OMRON Healthcare Europe NA, Hoofddorp, Netherlands). The measurements were performed in duplicate and after a resting time of at least 5 minutes. The averages were calculated for analysis. Core (tympanic) temperature was measured using a Genius™ 2 ear thermometer (Tyco Healthcare Group LP, Watertown, N.Y., USA). The mean arterial pressure (MAP) was calculated as diastolic blood pressure+(systolic blood pressure−diastolic blood pressure)/3. For the different autonomic response measure, the highest values (Eₘₐₓ) were used as outcome variable for the analysis because high cardiovascular stimulation or body temperature are the clinically relevant potentially adverse outcomes associated with MDMA use (Liechti, 2014; Liechti et al., 2005; Vizeli & Liechti, 2017).

The 5D-ASC scale (Dittrich, 1998; Studerus et al., 2010) was administered 6 hours after drug administration to retrospectively rate alterations in waking consciousness induced by MDMA.

Statistical Analyses

All data were analyzed using the R language and environment for statistical computing (R Core Team, 2019). Since some of the predictor and response variables contained missing data (TABLE 1), first multiple imputation (MI) was performed using the Multivariate Imputation via Chained Equations (MICE) package in R (Buuren & Groothuis-Oudshoorn, 2010). This method was chosen because it yields unbiased parameter estimates and standard errors under a “missing at random” (MAR) or “missing completely at random” (MCAR) missing data mechanism and maximizes statistical power by using all available information (Enders, 2010). The assumption of MAR was plausible in this study because the missing data mostly resulted from different study designs among the pooled studies. 20 imputations were generated of the missing values such that 20 completed datasets were obtained to protect against a potential power falloff from a too small number of imputations (Graham et al., 2007). The analyses of interest were then conducted in each completed data set and parameter estimates were pooled according to Rubin's rules (Little & Rubin, 2019), except for the LASSO models (see below).

TABLE 1

| Variable | Percent missing values |
|---|---|
| Independent variables | |
| Female | 0.0 |
| Age | 0.0 |
| Bodyweight | 0.0 |
| Drug dose | 0.0 |
| Number of times taken MDMA | 1.0 |
| Cytochrome (CYP) 2D6 activity score 3 | 2.6 |
| Trait Anxiety (STAI) | 14.9 |
| Activity (AMRS) | 0.0 |
| General Inactivation (AMRS) | 0.0 |
| Extroversion (AMRS) | 0.0 |
| Introversion (AMRS) | 0.0 |
| General Well-Being (AMRS) | 0.0 |
| Emotional Excitability (AMRS) | 0.0 |
| Anxiety-Depressiveness (AMRS) | 0.0 |
| Neuroticism (NEO-FFI) | 8.8 |
| Extroversion (NEO-FFI) | 8.8 |
| Openness to experience (NEO-FFI) | 8.8 |
| Agreeableness (NEO-FFI) | 8.8 |
| Conscientiousness (NEO-FFI) | 8.8 |
| Dependent variables | |
| MDMA plasma AUC | 0.5 |
| Heart rate | 0.0 |
| Mean arterial blood pressure | 0.0 |
| Body temperature | 0.0 |
| Activity (AMRS) | 0.0 |
| General inactivation (AMRS) | 0.0 |
| Extroversion (AMRS) | 0.0 |
| Extroversion (AMRS) | 0.0 |
| General Well-Being (AMRS) | 0.0 |
| Emotional Excitability (AMRS) | 0.0 |
| Anxiety-Depressiveness (AMRS) | 0.0 |
| Any drug effects (VAS) | 0.0 |
| Bad drug effects (VAS) | 0.0 |
| Good drug effects (VAS) | 0.0 |
| Closeness (VAS) | 0.0 |
| High mood (VAS) | 0.0 |
| Liking (VAS) | 0.0 |
| Openness (VAS) | 0.0 |
| Stimulated (VAS) | 0.0 |
| Talkative (VAS) | 0.0 |
| Oceanic Boundlessness (5D-ASC) | 2.1 |
| Dread of Ego Dissolution (5D-ASC) | 1.5 |
| Visionary Restructuralization (5D-ASC) | 1.5 |
| Impaired Control and Cognition (5D-ASC) | 1.5 |
| Anxiety (5D-ASC) | 1.5 |

To account for the clustering in the data arising from pooling across studies, linear mixed effects models were used in which the intercepts were allowed to vary randomly across studies. For each combination of predictor and response variable, an adjusted and unadjusted model was fitted using the R package nlme (Pinheiro et al., 2019). In the unadjusted model, only the predictor of interest was included in the fixed effects part of the model, whereas in the adjusted model “dose per body weight” was additionally included. Predictor and response variables were z-transformed before inclusion in the models, such that the estimated regression coefficients were standardized and comparable across predictors and responses. To account for multiple testing, p-values were corrected across all significance tests using the Benjamini-Hochberg procedure (Benjamini & Hochberg, 1995).

To identify the best subset of predictors for each response variables, the least absolute shrinkage and selection operator (LASSO) was applied using the R package penalized (Goeman, 2018). LASSO conducts both variable selection and regularization (i.e., shrinkage of regression coefficients) in order to optimize the predictive accuracy and interpretability of the model. It has been shown that variable selection with the LASSO is often more accurate than with traditional methods, such as stepwise methods (Tibshirani, 1997). For each response variable, a LASSO model was developed according to the following procedure. First, the optimal shrinkage parameter of each model was determined by performing grid search. For each lambda in the grid, bootstrapping with 50 iterations was performed and the average predictive performance (i.e. mean squared error) across all out-of-bag samples was calculated using the machine learning in R (mlr) package (Bischl et al., 2016). Second, the lambda value producing the highest out-of-bag predictive performance was chosen as the optimal lambda value and used for the final LASSO model fitted on the whole sample. Since it is currently unclear how to combine LASSO models across multiply imputed datasets and since the amount of missing data in our data set was relatively small, only single imputation was used for the LASSO models. Furthermore, for simplicity, a potential clustering was not accounted for in the data in these analyses.

Results

The most significant predictor variables for most of the MDMA response variables was the MDMA plasma concentration as shown in the unadjusted linear mixed effects models in FIG. 1. Smaller asterisks show non-corrected p-values. Bigger asterisks are p-values corrected for multiple testing across all 20*25=500 significance tests using the Benjamini-Hochberg procedure (Benjamini & Hochberg, 1995). *p<0.05, **p<0.01, ***p<0.001.

The most significant predictor variables for the blood plasma concentration of MDMA were the drug dose, body weight, sex, and genetically determined CYP2D6 activity (FIG. 1).

Specifically, MDMA plasma AUC over 6 hours is a measure of the overall exposure to the drug. MDMA plasma AUC is strongly and highly significantly correlated with the dose of MDMA (FIG. 1). MDMA plasma AUC is also strongly and highly significantly inversely correlated with the body weight of the subject ingesting the MDMA (FIG. 1). Furthermore, MDMA plasma AUC is also inversely correlated with the CYP2D6 activity (FIG. 1). Thus, persons with low CYP2D6 activity (defined genetically) have higher MDMA plasma levels. Finally, women exhibit significantly higher MDMA plasma AUC than men if MDMA is dosed with similar total doses of MDMA and not accounting for body weight.

Taken together, the data (FIG. 1) shows that the parameters linked to the MDMA exposure (MDMA plasma AUC, drug dose per body weight, and secondarily CYP2D6 activity) are the predictors most strongly and most significantly influencing the response to MDMA including subjective feelings (VASs) of any drug effects, good drug effects, high mood, liking, stimulated, Introversion (AMRS) and Oceanic Boundlessness (5D-ASC) as well as elevation of the mean arterial blood pressure.

Consistent with the above, FIG. 3 shows that the dose per body weight is the strongest predictor for the following outcome effects after MDMA administration: MDMA plasma AUC, mean arterial blood pressure (MAP) increase, introversion (AMRS), emotional excitability (AMRS), anxiety (AMRS), any drug effect (VAS), good drug effect (VAS), high mood (VAS), liking (VAS), stimulated (VAS), Oceanic Boundlessness (5D-ASC), and Impaired Control and Cognition (5D-ASC). The LASSO model was applied to identify the best subset of predictors for each response variables (Goeman, 2018).

The analysis showed that older age was the best predictor of a lower heart rate increase and lower temperature increase following MDMA and a higher bad drug effect (FIG. 3). This means that increases in blood pressure are more likely in elderly compared to increases in heart rate.

The finding that MDMA exposure in the body or measures linked to this (MDMA dose and body weight and CYP2D6 activity) mostly predict the acute response to MDMA is clinically important and shows that factors of set and setting are less relevant in predicting the response than the dose of MDMA. Thus, it is postulated that correct dosing per body weight within a controlled setting is critical to produce positive acute effects and also more critical than factors relating to set and setting. As a consequence, it is postulated that the present invention allows to maximize the positive effects of MDMA primarily by determining the correct dose to be used based on body weight and sex and CYP2D6 activity of the patient.

The finding of a relation between dose and good drug effect (within the range of doses tested here) shows that a high dose of MDMA (>75 mg) is needed to produce greater positive effects.

Because the MDMA plasma concentration is a variable that becomes known only after drug administration, it was classified as a response variable rather than a predictor variable for the further mixed effects model analysis.

Since the MDMA dose per body weight has an influence on the response variables via the MDMA plasma level, we adjusted the results with MDMA dose per body weight to see MDMA dose-independent predictors.

The dose-adjusted results are displayed in FIG. 2. Smaller asterisks show non-corrected p-values. Bigger asterisks are p-values corrected for multiple testing across all 17*25=425 significance tests using the Benjamini-Hochberg procedure (Benjamini & Hochberg, 1995). *p<0.05, **p<0.01, ***p<0.001.

Sex lost its predictor value when adjusting for dose and body weight (FIG. 2).

The genetically determined CYP2D6 activity still inversely correlated with the MDMA plasma concentration (p<0.01) (FIG. 2). Additionally, the inverse correlation between heart rate and age remained significant (p<0.05) (FIG. 2).

“Openness to experience” in the NEO-FFI positively correlated with VAS ratings of “closeness” and 5D-ASC ratings of “oceanic boundlessness” and “visionary restructuralization” after MDMA (p<0.05, p<0.05, and p<0.01, respectively) (FIG. 2).

Subjects who scored higher in “neuroticism” (NEO-FFI) and “trait anxiety” (STAI-T) were more likely to experience “dread of ego dissolution” and “impaired control and cognition” in the 5D-ASC (both p<0.01 and both p<0.05, respectively) (FIG. 2).

Further, past use of MDMA within the range of 0-5 had no significant effect on the response to MDMA (FIG. 2 and FIG. 3). This means that there was no marked difference in persons having no or a few previous experiences (up to 5).

Further, well-being (AMRS) before MDMA had no significant effect as predictor variable on the response to MDMA. This means that regardless of current mood, MDMA produces relatively similar effects and there is no need to be very well to experience more wellbeing after MDMA (FIG. 2). This is an important factor to be considered within the presently described invention of predicting positive responses to MDMA.

The most important predictors for each response variable of the multivariate analysis are shown in FIG. 3 by using the LASSO model.

Limitations

Limitations of the present study and invention include, first of all, the young, mostly MDMA-naïve, healthy study population. Thus, the findings only partly translate to psychiatric patients showing clearly greater psychopathology and presumably a greater likelihood of adverse psychological responses to MDMA.

Second, only two doses were examined and most of the subjects received the 125 mg MDMA dose that is also commonly used in clinical trials with MDMA.

Additionally, adverse events were not studied as they are relatively rare after MDMA administration in a controlled experimental setting (Hysek et al., 2014b; Kirkpatrick et al., 2014; Vollenweider et al., 2002), less amenable to an analysis as the present one, and also uncommonly reported in studies with patients (Mithoefer et al., 2010; Oehen et al., 2013).

Further, the “physical and social environment” may be important but showed little variation in the present study (Hartogsohn, 2016; Leary et al., 1963).

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Barrett F S, Johnson M W, & Griffiths R R (2017). Neuroticism is associated with challenging experiences with psilocybin mushrooms. Pers Individ Dif 117: 155-160.
2. Bedi G, & de Wit H (2011). Individual differences in acute responses to MDMA in humans: effects of sex and past ecstasy use. The Open Addiction Journal 4.
3. Benjamini Y, & Hochberg Y (1995). Controlling the false discovery rate: a practical and powerful approach to multiple testing. Journal of the Royal statistical society: series B (Methodological) 57: 289-300.
4. Bershad A K, Weafer J J, Kirkpatrick M G, Wardle M C, Miller M A, & de Wit H (2016). Oxytocin receptor gene variation predicts subjective responses to MDMA. Soc Neurosci 11: 592-599.
5. Bischl B, Lang M, Kotthoff L, Schiffner J, Richter J, Studerus E, Casalicchio G, & Jones Z M (2016). mlr: Machine Learning in R. The Journal of Machine Learning Research 17: 5938-5942.
6. Borkenau P, & Ostendorf F (eds) (2008). NEO-Fünf-Faktoren-Inventar (NEO-FFI) nach Costa and McCrae. Hogrefe: Göttingen.
7. Buuren Sv, & Groothuis-Oudshoorn K (2010). mice: Multivariate imputation by chained equations in R. Journal of statistical software: 1-68.
8. de la Torre R, Farre M, Mathuna B O, Roset P N, Pizarro N, Segura M, Torrens M, Ortuno J, Pujadas M, & Cami J (2005). MDMA (ecstasy) pharmacokinetics in a CYP2D6 poor metaboliser and in nine CYP2D6 extensive metabolisers. Eur J Clin Pharmacol 61: 551-554.
9. de la Torre R, Yubero-Lahoz S, Pardo-Lozano R, & Farre M (2012). MDMA, methamphetamine, and CYP2D6 pharmacogenetics: what is clinically relvant? Front Genet 3: 235.
10. Dittrich A (1998). The standardized psychometric assessment of altered states of consciousness (ASCs) in humans. Pharmacopsychiatry 31 (Suppl 2): 80-84.
11. Dolder P C, Muller F, Schmid Y, Borgwardt S J, & Liechti M E (2018). Direct comparison of the acute subjective, emotional, autonomic, and endocrine effects of MDMA, methylphenidate, and modafinil in healthy subjects. Psychopharmacology 235: 467-479.
12. Enders C K (2010) Applied missing data analysis. Guilford Press: New York.
13. Goeman J J (2018). Penalized R package, version 0.9-51.
14. Graham J W, Olchowski A E, & Gilreath T D (2007). How many imputations are really needed? Some practical clarifications of multiple imputation theory. Prevention science: the official journal of the Society for Prevention Research 8: 206-213.
15. Haijen E, Kaelen M, Roseman L, Timmermann C, Kettner H, Russ S, Nutt D, Daws R E, Hampshire A D G, Lorenz R, & Carhart-Harris R L (2018). Predicting Responses to Psychedelics: A Prospective Study. Front Pharmacol 9: 897.
16. Hartogsohn I (2016). Set and setting, psychedelics and the placebo response: An extra-pharmacological perspective on psychopharmacology. J Psychopharmacol 30: 1259-1267.
17. Harvanko A, Martin C, Lile J, Kryscio R, & Kelly T H (2016). Individual differences in the reinforcing and subjective effects of d-amphetamine: Dimensions of impulsivity. Experimental and clinical psychopharmacology 24: 436-446.
18. Hicks J K, Swen J J, Thorn C F, Sangkuhl K, Kharasch E D, Ellingrod V L, Skaar T C, Muller D J, Gaedigk A, Stingl J C, & Clinical Pharmacogenetics Implementation C (2013). Clinical pharmacogenetics implementation consortium guideline for CYP2D6 and CYP2C19 genotypes and dosing of tricyclic antidepressants. Clinical pharmacology and therapeutics 93: 402-408.
19. Holze F, Vizeli P, Muller F, Ley L, Duerig R, Varghese N, Eckert A, Borgwardt S, & Liechti M E (2020). Distinct acute effects of LSD, MDMA, and D-amphetamine in healthy subjects. Neuropsychopharmacology 45: 462-471.
20. Hysek C M, Brugger R, Simmler L D, Bruggisser M, Donzelli M, Grouzmann E, Hoener M C, & Liechti M E (2012a). Effects of the alpha2-adrenergic agonist clonidine on the pharmacodynamics and pharmacokinetics of 3,4-methylenedioxymethamphetamine in healthy volunteers. J Pharmacol Exp Ther 340: 286-294.
21. Hysek C M, & Liechti M E (2012). Effects of MDMA alone and after pretreatement with reboxetine, duloxetine, clonidine, carvedilol, and doxazosin on pupillary light reflex. Psychopharmacology 224: 363-376.
22. Hysek C M, Schmid Y, Rickli A, Simmler L D, Donzelli M, Grouzmann E, & Liechti M E (2012b). Carvedilol inhibits the cardiostimulant and thermogenic effects of MDMA in humans. Br J Pharmacol 166: 2277-2288.
23. Hysek C M, Schmid Y, Simmler L D, Domes G, Heinrichs M, Eisenegger C, Preller K H, Quednow B B, & Liechti M E (2014a). MDMA enhances emotional empathy and prosocial behavior. Soc Cogn Affect Neurosci 9: 1645-1652.
24. Hysek C M, Simmler L D, Ineichen M, Grouzmann E, Hoener M C, Brenneisen R, Huwyler J, & Liechti M E (2011). The norepinephrine transporter inhibitor reboxetine reduces stimulant effects of MDMA (“ecstasy”) in humans. Clinical pharmacology and therapeutics 90: 246-255.
25. Hysek C M, Simmler L D, Nicola V, Vischer N, Donzelli M, Krähenbühl S, Grouzmann E, Hoener M C, & Liechti M E (2012c). Duloxetine inhibits effects of MDMA (“ecstasy”) in vitro and in humans in a randomized placebo-controlled laboratory study. PLoS One 7: e36476.
26. Hysek C M, Simmler L D, Schillinger N, Meyer N, Schmid Y, Donzelli M, Grouzmann E, & Liechti M E (2014b). Pharmacokinetic and pharmacodynamic effects of methylphenidate and MDMA administered alone and in combination. Int J Neuropsychopharmacol 17: 371-381.
27. Janke W, & Debus G (1978) Die Eigenschaftswörterliste. Hogrefe: Göttingen.
28. Kirkpatrick M G, Baggott M J, Mendelson J E, Galloway G P, Liechti M E, Hysek C M, & de Wit H (2014). MDMA effects consistent across laboratories. Psychopharmacology 231: 3899-3905.
29. Leary T, Litwin G H, & Metzner R (1963). Reactions to Psilocybin Administered in a Supportive Environment. J Nery Ment Dis 137: 561-573.
30. Liechti M E (2014). Effects of MDMA on body temperature in humans. Temperature 1: 179-187.
31. Liechti M E, Gamma A, & Vollenweider F X (2001). Gender differences in the subjective effects of MDMA. Psychopharmacology 154: 161-168.
32. Liechti M E, Kunz I, & Kupferschmidt H (2005). Acute medical problems due to Ecstasy use: case-series of emergency department visits. Swiss Med Wkly 135: 652-657.
33. Little R J, & Rubin D B (2019) Statistical analysis with missing data. vol. 793. Wiley.
34. MacLean K A, Johnson M W, & Griffiths R R (2011). Mystical experiences occasioned by the hallucinogen psilocybin lead to increases in the personality domain of openness. J Psychopharmacol 25: 1453-1461.
35. Mithoefer M C, Feduccia A A, Jerome L, Mithoefer A, Wagner M, Walsh Z, Hamilton S, Yazar-Klosinski B, Emerson A, & Doblin R (2019). MDMA-assisted psychotherapy for treatment of PTSD: study design and rationale for phase 3 trials based on pooled analysis of six phase 2 randomized controlled trials. Psychopharmacology 236: 2735-2745.
36. Mithoefer M C, Wagner M T, Mithoefer A T, Jerome I, & Doblin R (2010). The safety and efficacy of ±3,4-methylenedioxymethamphetamine-assisted psychotherapy in subjects with chronic, treatment-resistant posttraumatic stress disorder: the first randomized controlled pilot study. J Psychopharmacol 25: 439-452.
37. Oehen P, Traber R, Widmer V, & Schnyder U (2013). A randomized, controlled pilot study of MDMA (±3,4-methylenedioxymethamphetamine)-assisted psychotherapy for treatment of resistant, chronic post-traumatic stress disorder (PTSD). J Psychopharmacol 27: 40-52.
38. Ormel J, Riese H, & Rosmalen J G (2012). Interpreting neuroticism scores across the adult life course: immutable or experience-dependent set points of negative affect? Clin Psychol Rev 32: 71-79.
39. Pardo-Lozano R, Farre M, Yubero-Lahoz S, O'Mathuna B, Torrens M, Mustata C, Perez-Mana C, Langohr K, Cuyas E, Carbo M, & de la Torre R (2012). Clinical pharmacology of 3,4-methylenedioxymethamphetamine (MDMA, “ecstasy”): the influence of gender and genetics (CYP2D6, COMT, 5-HTT). PLoS One 7: e47599.
40. Pinheiro J, Bates D, DebRoy S, Sarkar D, & R Core Team (2019). nlme: Linear and Nonlinear Mixed Effects Models. R package version 3.1-140.
41. R Core Team (2019). R: A language and environment for statistical computing.
42. Roberti J W (2004). A review of behavioral and biological correlates of sensation seeking. J Res Pers 38: 256-279.
43. Schmid Y, Hysek C M, Preller K H, Bosch O G, Bilderbeck A C, Rogers R D, Quednow B B, & Liechti M E (2015a). Effects of methylphenidate and MDMA on appraisal of erotic stimuli and intimate relationships. European neuropsychopharmacology: the journal of the European College of Neuropsychopharmacology 25: 17-25.
44. Schmid Y, Hysek C M, Simmler L D, Crockett M J, Quednow B B, & Liechti M E (2014). Differential effects of MDMA and methylphenidate on social cognition. J Psychopharmacol 28: 847-856.
45. Schmid Y, & Liechti M E (2018). Long-lasting subjective effects of LSD in normal subjects. Psychopharmacology 235: 535-545.
46. Schmid Y, Rickli A, Schaffner A, Duthaler U, Grouzmann E, Hysek C M, & Liechti M E (2015b). Interactions between bupropion and 3,4-methylenedioxymethamphetamine in healthy subjects. J Pharmacol Exp Ther 353: 102-111.
47. Schmid Y, Vizeli P, Hysek C M, Prestin K, Meyer Zu Schwabedissen H E, & Liechti M E (2016). CYP2D6 function moderates the pharmacokinetics and pharmacodynamics of 3,4-methylene-dioxymethamphetamine in a controlled study in healthy individuals. Pharmacogenet Genomics 26: 397-401.
48. Simmler L D, Hysek C M, & Liechti M E (2011). Sex differences in the effects of MDMA (ecstasy) on plasma copeptin in healthy subjects. The Journal of clinical endocrinology and metabolism 96: 2844-2850.
49. Spielberger C D, Gorsuch R C, & Lusheme R E (1970) Manual for the Stait Trait Anxiety Inventory. Consulting Psychologists Press: Palo Alto.
50. Studerus E, Gamma A, Kometer M, & Vollenweider F X (2012). Prediction of psilocybin response in healthy volunteers. PLoS One 7: e30800.
51. Studerus E, Gamma A, & Vollenweider F X (2010). Psychometric evaluation of the altered states of consciousness rating scale (OAV). PLoS One 5: e12412.
52. Tibshirani R (1997). The lasso method for variable selection in the Cox model. Statistics in medicine 16: 385-395.
53. Vizeli P, & Liechti M E (2017). Safety pharmacology of acute MDMA administration in healthy subjects. J Psychopharmacol 31: 576-588.
54. Vizeli P, & Liechti M E (2018). Oxytocin receptor gene variations and socio-emotional effects of MDMA: A pooled analysis of controlled studies in healthy subjects. PLoS One 13: e0199384.
55. Vizeli P, Meyer Zu Schwabedissen H E, & Liechti M E (2018). No major role of norepinephrine transporter gene variations in the cardiostimulant effects of MDMA. Eur J Clin Pharmacol 74: 275-283.
56. Vizeli P, Meyer Zu Schwabedissen H E, & Liechti M E (2019). Role of Serotonin Transporter and Receptor Gene Variations in the Acute Effects of MDMA in Healthy Subjects. ACS Chem Neurosci 10: 3120-3131.
57. Vizeli P, Schmid Y, Prestin K, Meyer zu Schwabedissen H E, & Liechti M E (2017). Pharmacogenetics of ecstasy: CYP1A2, CYP2C19, and CYP2B6 polymorphisms moderate pharmacokinetics of MDMA in healthy subjects. European neuropsychopharmacology: the journal of the European College of Neuropsychopharmacology 27: 232-238.
58. Vollenweider F X, Liechti M E, Gamma A, Greer G, & Geyer M (2002). Acute psychological and neurophysiological effects of MDMA in humans. J Psychoactive Drugs 34: 171-184.
59. Wagner M T, Mithoefer M C, Mithoefer A T, MacAulay R K, Jerome L, Yazar-Klosinski B, & Doblin R (2017). Therapeutic effect of increased openness: Investigating mechanism of action in MDMA-assisted psychotherapy. J Psychopharmacol 31: 967-974.
60. White T L (2017). Beyond Sensation Seeking: A Conceptual Framework for Individual Differences in Psychostimulant Drug Effects in Healthy Humans. Curr Opin Behav Sci 13: 63-70.

What is claimed is:

1. A method of dosing an empathogen/entactogen in treating patients, including the steps of:
assessing patient characteristics before empathogen/entactogen use;
administering empathogen/entactogen to the patient based on the patient characteristics; and
producing maximum positive subjective acute effects in the patient.

2. The method of claim 1, wherein said administering step is performed in a situation chosen from the group consisting of a therapeutic situation or a legal controlled situation in healthy subjects.

3. The method of claim 1, wherein positive subjective acute effects are chosen from the group consisting of good drug effects, drug liking, trust, feelings of closeness, feeling open, oceanic boundlessness, experience of unity, spiritual experience, blissful state, insightfulness, connectedness, mystical experiences, mystical-type effects, positive mood, transcendence of time/space, ineffability, well-being, peak experience, and combinations thereof.

4. The method of claim 1, wherein patient characteristics are chosen from the group consisting of age, sex, drug dose, body weight, previous drug experience, genetics, personality, mood before intake, and combinations thereof.

5. The method of claim 1, wherein patient characteristics include cytochrome P450 2D6 activity, and adjusting a dose based on patients with poor CYP2D6 activity exhibiting higher MDMA concentrations and a stronger MDMA response compared with CYP2D6 extensive metabolizers.

6. The method of claim 1, wherein the empathogen/entactogen is chosen from the group consisting of 3,4-methylenedioxymethamphetamine (MDMA), 3,4-methylenedioxyamphetamine (MDA), 3,4-methylenedioxyethylamphetamine (MDEA), 5,6-methylenedioxy-2-aminoindane (MDAI), mephedrone, methylone, 3-MMC, homologues thereof, analogues thereof, and prodrugs thereof.

7. The method of claim 6, wherein the empathogen/entactogen is MDMA and is administered in a dose of 20-200 mg.

8. A method of determining a dose of an empathogen/entactogen based on body weight, sex, and CYP2D6 activity, including the steps of:
assessing patient characteristics of body weight, sex, and CYP2D6 activity before empathogen/entactogen use;
administering the empathogen/entactogen to the patient based on the patient characteristics; and
producing maximum positive subjective acute effects in the patient.

9. The method of claim 8, wherein administering a high dose of empathogen/entactogen per body weight leads to a more intense response to empathogen/entactogen with more positive and more cardiostimulant effects.

10. The method of claim 8, wherein patients with low CYP2D6 activity have higher empathogen/entactogen plasma levels than patients with high CYP2D6 activity.

11. The method of claim 8, wherein women have higher empathogen/entactogen plasma AUC than men.

12. The method of claim 8, wherein positive subjective acute effects are chosen from the group consisting of good drug effects, drug liking, trust, feelings of closeness, feeling open, oceanic boundlessness, experience of unity, spiritual experience, blissful state, insightfulness, connectedness, mystical experiences, mystical-type effects, positive mood, transcendence of time/space, ineffability, well-being, peak experience, and combinations thereof.

13. The method of claim 8, wherein the empathogen/entactogen is chosen from the group consisting of 3,4-methylenedioxymethamphetamine (MDMA), 3,4-methylenedioxyamphetamine (MDA), 3,4-methylenedioxyethylamphetamine (MDEA), 5,6-methylenedioxy-2-aminoindane (MDAI), mephedrone, methylone, 3-MMC, homologues thereof, analogues thereof, and prodrugs thereof.

14. The method of claim 13, wherein the empathogen/entactogen is MDMA and is administered in a dose of 20-200 mg.

15. A method of refining dosing of an empathogen/entactogen, including the steps of:
using questionnaires including NEO-FFI, STAI-T, and AMRS scales with a patient;
evaluating questionnaire responses; and
refining dosing of the empathogen/entactogen in the patient based on the questionnaires.

16. The method of claim 15, wherein openness to experience on NEO-FFI predicts a more positive acute response, neuroticism on NEO-FFI predicts negative experiences, trait anxiety on STAI-T predicts negative experiences, and AMRS predicts likelihood of a negative acute effect.

17. The method of claim 15, wherein the empathogen/entactogen is chosen from the group consisting of 3,4-methylenedioxymethamphetamine (MDMA), 3,4-methylenedioxyamphetamine (MDA), 3,4-methylenedioxyethylamphetamine (MDEA), 5,6-methylenedioxy-2-aminoindane (MDAI), mephedrone, methylone, 3-MMC, homologues thereof, analogues thereof, and prodrugs thereof.

18. The method of claim 17, wherein the empathogen/entactogen is MDMA and is administered in a dose of 20-200 mg.

19. A method of predicting future dosing with an empathogen/entactogen, including the steps of:
measuring plasma concentrations of the empathogen/entactogen in a patient after administration of a dose of the empathogen/entactogen;
adjusting the dose of the empathogen/entactogen in the patient, thereby optimizing the positive response to the empathogen/entactogen, and optimizing efficacy and safety of the empathogen/entactogen treatment.

20. The method of claim 19, wherein the empathogen/entactogen is chosen from the group consisting of 3,4-methylenedioxymethamphetamine (MDMA), 3,4-methylenedioxyamphetamine (MDA), 3,4-methylenedioxyethylamphetamine (MDEA), 5,6-methylenedioxy-2-aminoindane (MDAI), mephedrone, methylone, 3-MMC, homologues thereof, analogues thereof, and prodrugs thereof.

21. The method of claim 20, wherein the empathogen/entactogen is MDMA and is administered in a dose of 20-200 mg.

22. A method of evaluating feasibility of patients to receive an empathogen/entactogen as treatment, including the steps of:
assessing patient characteristics; and
evaluating feasibility of the patient to receive the empathogen/entactogen as a treatment.

23. The method of claim 22, wherein patient characteristics are chosen from the group consisting of age, sex, drug dose, body weight, previous drug experience, genetics, personality, mood before intake, and combinations thereof.

24. The method of claim 22, wherein the empathogen/entactogen is chosen from the group consisting of 3,4-methylenedioxymethamphetamine (MDMA), 3,4-methylenedioxyamphetamine (MDA), 3,4-methylenedioxyethylamphetamine (MDEA), 5,6-methylenedioxy-2-aminoindane (MDAI), mephedrone, methylone, 3-MMC, homologues thereof, analogues thereof, and prodrugs thereof.

25. The method of claim 24, wherein the empathogen/entactogen is MDMA and is administered in a dose of 20-200 mg.

26. A method of optimizing empathogen/entactogen treatment in a patient, including the steps of:
assessing openness in the patient prior to empathogen/entactogen use;
predicting a positive response leading to more openness; and
optimizing empathogen/entactogen treatment including repeated administration in different therapeutic sessions to lead to greater openness and a greater therapeutic response over time.

27. The method of claim 26, wherein the empathogen/entactogen is chosen from the group consisting of 3,4-methylenedioxymethamphetamine (MDMA), 3,4-methylenedioxyamphetamine (MDA), 3,4-methylenedioxyethylamphetamine (MDEA), 5,6-methylenedioxy-2-aminoindane (MDAI), mephedrone, methylone, 3-MMC, homologues thereof, analogues thereof, and prodrugs thereof.

28. The method of claim 27, wherein the empathogen/entactogen is MDMA and is administered in a dose of 20-200 mg.
